(19) United States
(12) Patent Application Publication
(10) Pub. No.: US 2020/0163568 A1
(43) Pub. Date: May 28, 2020

(54) METHOD AND SYSTEM FOR DETERMINING CONFIDENCE LEVEL OF A PERSON USING ELECTROENCEPHALOGRAM

(71) Applicant: Tata Consultancy Services Limited, Mumbai (IN)

(72) Inventors: Rahul Dasharath GAVAS, Kolkata (IN); Debatri CHATTERJEE, Kolkata (IN); Aniruddha SINHA, Kolkata (IN); Anirban CHOWDHURY, Kolkata (IN); Sanjoy Kumar SAHA, Kolkata (IN)

(73) Assignee: Tata Consultancy Services Limited, Mumbai (IN)

(21) Appl. No.: 16/663,176

(22) Filed: Oct. 24, 2019

(30) Foreign Application Priority Data
Oct. 26, 2018 (IN) 201821040568

(51) Int. Cl. A61B 5/04; A61B 5/00; A61B 5/16; A61B 5/0484

(52) U.S. Cl. CPC A61B 5/04014; A61B 5/6803; A61B 5/7239; A61B 5/04842; A61B 5/167

(57) ABSTRACT

Metacognitive confidence is defined as the confidence generated from the observation and critical analysis of one's own the decision making process. There are various studies indicative of the importance of measurement of confidence level of the person while doing a task. The existing confidence level measurement methods provide various limitations such invasive and complex experimental setup, noise and artifacts in the signal. A system and method for determining confidence level of a person using electroencephalogram has been provided. The system is configured to build a metric to determine the amount of metacognitive confidence, in presence of different cognitive load condition, directly from brain activity using electroencephalogram signals. The brain activity acquired from the frontal and temporal part of the brain at different frequency bands and combined with suitable weights to form the confidence metric.

### PRIORITY CLAIM

This U.S. patent application claims priority under 35 U.S.C. § 119 to: India Application No. 201821040568, filed on Oct. 26, 2018. The entire contents of the aforementioned application are incorporated herein by reference.

### TECHNICAL FIELD

The embodiments herein generally relates to the field of confidence level measurement. More particularly, but not specifically, the invention provides a system and method for determining confidence level of a person using electroencephalogram (EEG) of the person.

### BACKGROUND

Majority of the day-to-day decisions are associated with a sense of confidence. Even in the absence of explicit feedback, we possess an awareness of the goodness of the decisions made. Assessment of confidence is crucial as it is a major indicator of cognitive impairments like obsessive-compulsive disorder (OCD) and anxiety. A “checking” behavior is seen in subjects with reduced confidence level in their own memory. Thus confidence or the capability of being aware of the goodness of the self-performance is vital for guiding adaptive behavior in cases which lack direct feedback from the surroundings.

Metacognitive confidence is defined as the confidence generated from the observation and critical analysis of one's own the decision making process. The metacognitive confidence is very significant when in assessing the accuracy of our thinking and decision making process, while any external feedback from the environment is not present.

The metacognitive confidence governs the process of our response to a situation by pondering on whether to act immediately, or to wait and gather further evidence before deciding to act, or modify the existing world model with newly found evidences. Indeed, the higher is the level of uncertainty in the external events in the environment, the higher is the need to be attentive in information gathering to boost the learning process.

The evaluation or determination of confidence level of the person is an important from various points of view. Various studies have been published in the art to indicate the importance of the metacognitive confidence. Various methods have been used also to determine the confidence level of the person based on visual stimulation. Though there are some limitations regarding the metacognitive confidence estimation that need to be overcome. The pupillometry may get affected by the changes in the visual stimulation. The confidence makers based upon the neural activity, developed so far depends on the invasive recordings of the single neuron activity which is not feasible to build a practical instrument for confidence measurement, which could be used in a real world scenario.

### SUMMARY

The following presents a simplified summary of some embodiments of the disclosure in order to provide a basic understanding of the embodiments. This summary is not an extensive overview of the embodiments. It is not intended to identify key/critical elements of the embodiments or to delineate the scope of the embodiments. Its sole purpose is to present some embodiments in a simplified form as a prelude to the more detailed description that is presented below.

In view of the foregoing, an embodiment herein provides a system for determining confidence level of a person using electroencephalogram (EEG). The system comprises a display screen, an EEG sensor, a memory and a processor. The display screen present in front of the person to provide a stimulus. The EEG sensor attached on the person configured to capture electroencephalogram (EEG) signal of the person in response to the stimulus, wherein the EEG sensor is using a predefined number of electrodes for sensing. The processor further comprises a filtering module, an independent component analysis module, a reconstruction module, a decomposition module, a band power calculation module, a band power feature vector generation module, an outlier removal module, a cluster determination module, a normalized distance calculation module, a relevant feature identification module and a confidence metric generation module. The filtering module filters the captured EEG signal using a band pass filter. The independent component analysis module performs an independent component analysis (ICA) on the filtered EEG signal to remove the artifacts generated due to eye blink of the person. The reconstruction module reconstructs the filtered EEG signal after removing the artifacts. The decomposing module decomposes the reconstructed EEG signal into three frequency bands. The band power calculation module calculates band powers corresponding to each of the three frequency bands. The band power feature vector generation module generates band power feature vectors corresponding to each of a plurality of elements, wherein the plurality of elements are decided based on the predefined number of electrodes and the three frequency bands, wherein the generated band power feature vectors are represented in a feature matrix. The outlier removal module removes outliers from the feature matrix using a random sample consensus (RANSAC) method, wherein the removal results in the generation of an inliers feature matrix. The cluster determination module determines a first cluster corresponding to a high confidence condition and a second cluster corresponding to a low confidence condition for the each of the feature vectors of the inlier feature matrix. The normalized distance calculation module calculates a normalized distance between the first cluster and the second cluster for each feature vectors, wherein the normalized distance results in the generation of a normalized distance matrix. The relevant feature identification module identifies a set of relevant features with maximum separability using the normalized distance matrix based on a predefined condition involving features having statistically significant p-value difference between the low confidence and the high confidence condition. The confidence metric generation module generates a confidence metric by taking average of the set of relevant features to determine the confidence level of the person.

In another aspect the embodiment here provides a method for determining confidence level of a person using electroencephalogram (EEG). Initially, a stimulus is provided to the person using a display screen present in front of the person. In the next step, electroencephalogram (EEG) signal of the person is captured in response to the stimulus using an EEG sensor attached on the person, wherein the EEG sensor is using a predefined number of electrodes for sensing. Further, the captured EEG signal is filtered using a band pass filter. Later and an independent component analysis (ICA) is performed on the filtered EEG signal to remove the artifacts generated due to eye blink of the person. In the next step, the filtered EEG signal is reconstructed after removing the artifacts. In the next step, the reconstructed EEG signal is decomposed into three frequency bands. Band powers are then calculated corresponding to each of the three frequency bands. In the next step, band power feature vectors are generated corresponding to each of a plurality of elements, wherein the plurality of elements are decided based on the predefined number of electrodes and the three frequency bands, wherein the generated band power feature vectors are represented in a feature matrix. Further, outliers are removed from the feature matrix using a random sample consensus (RANSAC) method, wherein the removal results in the generation of an inliers feature matrix. In the next step, a first cluster corresponding to a high confidence condition and a second cluster corresponding to a low confidence condition are determined for the each of the feature vectors of the inlier feature matrix. Further, a normalized distance is calculated between the first cluster and the second cluster for each feature vectors, wherein the normalized distance results in the generation of a normalized distance matrix. In the next step, a set of relevant features are identified with maximum separability using the normalized distance matrix based on a predefined condition involving features having statistically significant p-value difference between the low confidence and the high confidence condition. And finally, a confidence metric is generated by taking average of the set of relevant features to determine the confidence level of the person.

In yet another embodiment, one or more non-transitory machine readable information storage mediums comprising one or more instructions is provided. The one or more instructions when executed by one or more hardware processors causes the one or more hardware processors to perform a method for providing a stimulus to the person using a display screen present in front of the person; capturing electroencephalogram (EEG) signal of the person in response to the stimulus using an EEG sensor attached on the person, wherein the EEG sensor is using a predefined number of electrodes for sensing; filtering the captured EEG signal using a band pass filter; performing an independent component analysis (ICA) on the filtered EEG signal to remove the artifacts generated due to eye blink of the person; reconstructing the filtered EEG signal after removing the artifacts; decomposing the reconstructed EEG signal into three frequency bands; calculating band powers corresponding to each of the three frequency bands; generating band power feature vectors corresponding to each of a plurality of elements, wherein the plurality of elements are decided based on the predefined number of electrodes and the three frequency bands, wherein the generated band power feature vectors are represented in a feature matrix; removing outliers from the feature matrix using a random sample consensus (RANSAC) method, wherein the removal results in the generation of an inliers feature matrix; determining a first cluster corresponding to a high confidence condition and a second cluster corresponding to a low confidence condition for the each of the feature vectors of the inlier feature matrix; calculating a normalized distance between the first cluster and the second cluster for each feature vectors, wherein the normalized distance results in the generation of a normalized distance matrix; identifying a set of relevant features with maximum separability using the normalized distance matrix based on a predefined condition involving features having statistically significant p-value difference between the low confidence and the high confidence condition; and generating a confidence metric by taking average of the set of relevant features to determine the confidence level of the person.

It should be appreciated by those skilled in the art that any block diagram herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and so executed by a computing device or processor, whether or not such computing device or processor is explicitly shown.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 illustrates a block diagram of a system for determining confidence level of a person using electroencephalogram according to an embodiment of the present disclosure;

FIG. 2 shows an experimental setup of the system for determining confidence level of the person using electroencephalogram according to an embodiment of the disclosure;

FIG. 3A-3C is a flowchart illustrating the steps involved in determining confidence level of a person using electroencephalogram according to an embodiment of the present disclosure;

FIG. 4 shows a schema of an addition task stimulus according to an embodiment of the disclosure;

FIG. 5 shows a schema of an anagram task according to an embodiment of the disclosure;

FIGS. 6A and 6B shows a boxplot for the features values of Theta_Fp2 for high condition and low condition classes according to an embodiment of the disclosure;

FIG. 7 shows mean and standard deviation of the high condition and low condition clusters for each feature types according to an embodiment of the disclosure; and

FIG. 8 shows separation between the distributions of the HC and LC from CFM formulation according to an embodiment of the disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims.

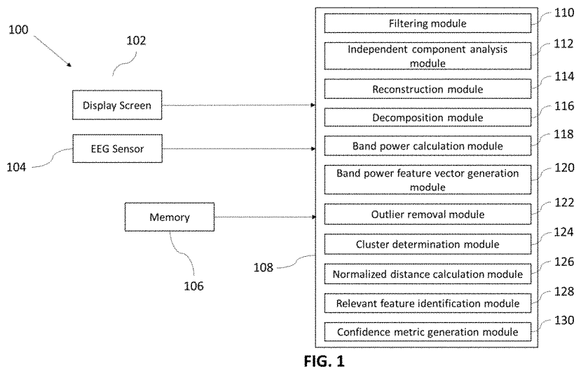

Referring now to the drawings, and more particularly to FIG. 1 through FIG. 8, where similar reference characters denote corresponding features consistently throughout the figures, there are shown preferred embodiments and these embodiments are described in the context of the following exemplary system and/or method.

According to an embodiment of the disclosure, a system 100 for determining confidence level of a person using electroencephalogram (EEG) of the person is shown in the block diagram of FIG. 1. The system 100 is configured to determine the confidence level of the person while the person is performing a task. The system 100 is used to estimate a metacognitive confidence metric based on the EEG signal, which can measure the brain activity in a non-invasive way. The confidence metric was assigned to the individual trials of two different cognitive task (Anagram task and Number Addition task which are considered as high confidence (HC) and low confidence (LC) respectively) independent of the cognitive load (CL) condition, be it high load or low load). The system and method computes a single value (scalar) measurement of the metacognitive confidence associated with the cognitive task based on a particular combination of the band power measurements of EEG acquired from different channels.

According to an embodiment of the disclosure, the system 100 further comprises a display screen 102 or an input/output module 102, an electroencephalogram sensor 104, a memory 106 and a processor 108 as shown in the block diagram of FIG. 1. The processor 108 works in communication with the memory 106. The processor 108 further comprises a plurality of modules. The plurality of modules accesses the set of algorithms stored in the memory 106 to perform a certain functions. The processor 108 further comprises a filtering module 110, an independent component analysis module 112, a reconstruction module 114, a decomposition module 116, a band power calculation module 118, a band power feature vector generation module 120, an outlier removal module 122, a cluster determination module 124, a normalized distance calculation module 126, a relevant feature identification module 128 and a confidence metric generation module 130.

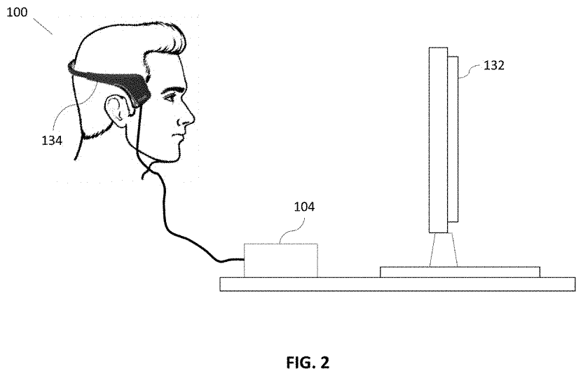

According to an embodiment of the disclosure the input/output module 102 or a display screen 102 is configured to provide a stimulus to the persons. The input/output module 102 is configured to provide one or more task to the person. In an example of the disclosure, the input/output module 102 is a computer screen 132 as shown in the experimental setup of FIG. 2. In the present example, the input/output module 102 is configured to provide an addition task and an anagram task to the person for determining the confidence level of the person. The input/output module 102 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite.

According to an embodiment of the disclosure, the system 100 is using a low cost we have used a low cost EEG headband from company Muse as the EEG sensor 104. The use of any other similar EEG sensor is well within the scope of this disclosure. The EEG sensor 104 has four dry electrodes corresponding to Fp1, Fp2, TP9 and TP10 with sampling frequency of 220 Hz. The reference electrode is at Fpz and DRLs are at a distance of one inch from the reference on both sides. The headband is worn like a pair of glasses, with the frontal electrodes placed over the middle of the forehead, and the rear electrodes placed behind the ears.

According to an embodiment of the disclosure the stimulus is presented on the display screen 102 with a resolution of 1366*768 pixels placed at a distance of about 60 cm from the person.

According to an embodiment of the disclosure, the system 100 comprises the filtering module 110. The filtering module 110 is configured to filter the captured EEG signal using a band pass filter. In an embodiment a 4th order band pass filter has been used. The raw EEG data x are generally contaminated by different noise sources and artifacts. Therefore data pre-processing is essential before going for the analysis of the EEG signal. The data preprocessing was done by filtering the x with a band pass filter with a pass band of about 0.5 to 40 Hz.

According to an embodiment of the disclosure, the system 100 further comprises the independent component analysis (ICA) module 112. The ICA module 112 is configured to perform an independent component analysis (ICA) on the filtered EEG signal. The ICA is performed to remove the artifacts generated due to eye blink of the person. The independent components (ICs) can be obtained from equation (1):

u=W*x (1)

where, u is the matrix containing the ICs. In an example, the raw EEG data x contains 4 channels and hence equation (1) decomposes x into 4 ICs. The ICs were visually inspected and the component containing the eye-blink was removed from the u, by replacing the values of that particular component with zeros.

In an example of the disclosure, the functions to perform the band pass filtering and ICA were used from EEGLAB toolbox for MATLAB. The runica function based on the infomax ICA algorithm was used to compute the un-mixing matrix W. The use of any other tool is well within the scope of this disclosure.

According to an embodiment of the disclosure, the system 100 comprises the reconstruction module 114. The reconstruction module 114 is configured to reconstruct the filtered EEG signal after removing the artifacts. After the removal of the artifact component x was reconstructed as y using equation (2)

y=W−1*u (2)

According to an embodiment of the disclosure, the system 100 also comprises the decomposition module 116 and the band power calculation module 118. The decomposition module 116 is configured to decompose the reconstructed EEG signal into three frequency bands. The three frequency bands are namely theta in the frequency range of about 4.5 to 7.5 Hz, alpha ion the frequency range of about 8 to 12.5 Hz and beta in the frequency range of about 13 to 30 Hz. Further, the band power calculation module 118 is configured to calculate the band powers corresponding to each of the three frequency bands. The band powers corresponding to each of these bands were calculated taking a 500 ms long epoch before the end time-point of each trial. This means that for the anagram task this time period is 500 ms before the mouse-click to the instant of the mouse-click and for the number addition task (which had a fixed trial period of 3 s) the time period was between 2.5 s to 3 s. The band pass filtering was done using 4th order butterworth filter. Each sample in the band-pass filtered time-period was squared and averaged over the entire period to get the band-power feature corresponding to each trial. This process was continued for all 4 EEG channels (TP9, Fp1, Fp2, and TP10).

According to an embodiment of the disclosure, the system 100 further comprises the band power feature vector generation module 120. The band power feature vector generation module 120 is configured to generate a band power feature vector corresponding to a plurality of elements. The plurality of elements are decided based on the predefined number of electrodes and the three frequency bands. Thus in the present example, after obtaining the band-powers, a band-power feature vector of 12 elements (3 frequency bands per 4 EEG channels) for all the trials comprising two different tasks for all the participants are obtained.

According to an embodiment of the disclosure, the system 100 also comprises the outlier removal module 122 and the cluster determination module 124. The outlier removal module 122 is configured to remove outliers from the feature matrix using a random sample consensus (RANSAC) method, wherein the removal results in the generation of an inliers feature matrix. Further, the cluster determination module 124 is configured to determine a first cluster corresponding to the high confidence condition (HC) and a second cluster corresponding to a low confidence (LC) condition for the each of the feature vectors of the inlier feature matrix. This has been done by plotting the boxplot of the data distribution of each feature. The example of the same has been provided in the later part of the disclosure.

According to an embodiment of the disclosure, the system 100 also comprises the normalized distance calculation module 126 and the relevant feature identification module 128. The normalized distance calculation module 126 is configured to calculate a normalized distance between the first cluster and the second cluster for each feature vectors, wherein the normalized distance results in the generation of a normalized distance matrix (Xn). The normalization of the distance of a particular feature value from a particular trial is calculated using the following pseudo code:

IF (feature value (Fi) falls into Cluster_HC)

THEN dist_Fi=1;

ELSE IF (feature value (Fi) falls into Cluster_LC)

THEN dist_Fi=0;

ELSE

dist=min(Fi_to_ Cluster_HC, Fi_to_Cluster_LC);
IF(dist is closer to Cluster_HC)
THEN dist=0.5+0.5*((std_of_Cluster_HC)/dist);
ELSE

dist=0.5*((std_of_Cluster_LC)/dist);

END

END

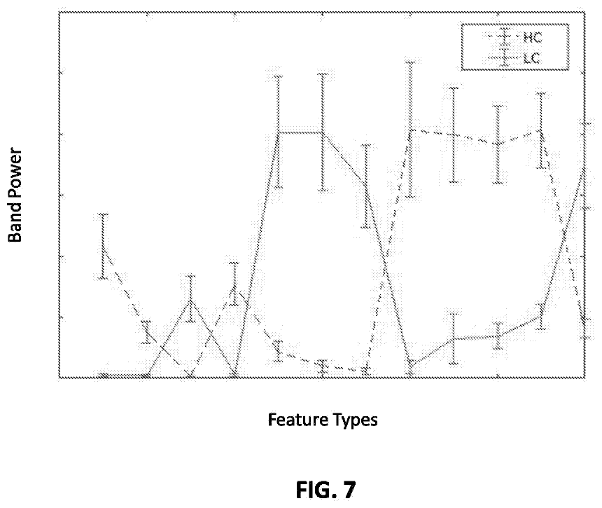

Thus the normalized distance matrix is obtained corresponding to HC and LC where it is expected in ideal case that all the values of the normalized distance matrix related to HC should be >0.5 and values related to LC should be <0.5. After this formulation it is seen that all the feature types which have statistically significant difference between the HC and LC groups have the same polarity. This can be seen from the Table 1 for all the significant feature types, namely: Theta_TP9, Theta_Fp2, Theta_TP10, Alpha_Fp2, Beta_TP9 and Beta_TP10 (with reference to FIG. 7 these features can be identified as the sequence number: 1, 3, 4, 7, 9, 12).

TABLE 1
THE SAMPLE MEAN AND GROUP
MEAN OF VARIOUS FEATURES

| | | Sample | Group | Difference | |
|---|---|---|---|---|---|
| Feature | Feature | Mean of | Mean | in Sample | |
| Seq | Name | HC | of LC | Mean | p-value |
| 1 | Theta_TP9 | 0.3595 | 0.261 | Positive | 0.0050 < 0.05 |
| 3 | Theta_Fp2 | 0.6811 | 0.3867 | Positive | 0.0000 < 0.05 |
| 4 | Theta_TP10 | 0.3466 | 0.2604 | Positive | 0.0131 < 0.05 |
| 7 | Alpha_Fp2 | 0.602 | 0.404 | Positive | 0.0000 < 0.05 |
| 9 | Beta_TP9 | 0.4896 | 0.3846 | Positive | 0.0109 < 0.05 |
| 12 | Beta_TP10 | 0.5586 | 0.4654 | Positive | 0.0195 < 0.05 |

The relevant feature identification module 128 is configured to identify a set of relevant features with maximum separability using the normalized distance matrix based on a predefined condition involving features having statistically significant p-value difference between the low confidence and the high confidence condition. Further, the F1-score is also used to determine the quality of separation.

The confidence metric generation module 130 is configured to generating a confidence metric by taking average of the set of relevant features. Therefore it was concluded that as the group mean difference between the sample means of the HC and LC classes are all positive (meaning that they have the same polarity), they can be averaged and get a scalar measurement of Confidence Metric (CFM) which can give significant separation between HC and LC. As per this argument CFM is defined as

$$CFM = \frac{\begin{matrix}ThetaTP 9 + ThetaFP 2 + ThetaTP 10 + \\ AlphaFP 2 + BetaTP 9 + BetaTP 10\end{matrix}}{6} \qquad ( 3 )$$

The confidence metric is then used to determine the confidence level of the person.

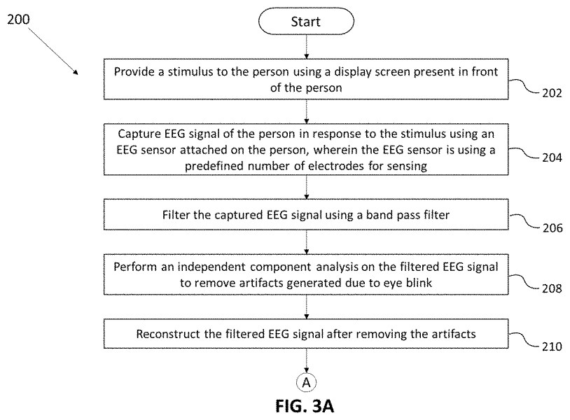

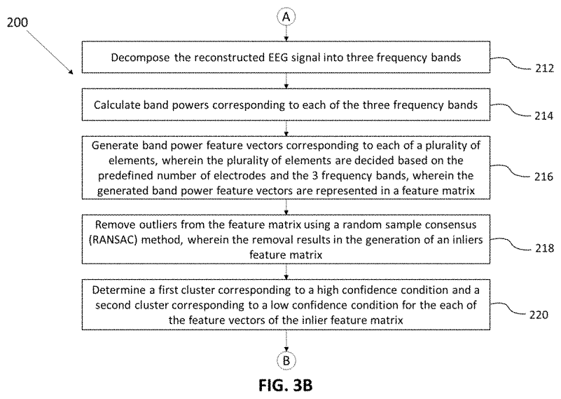

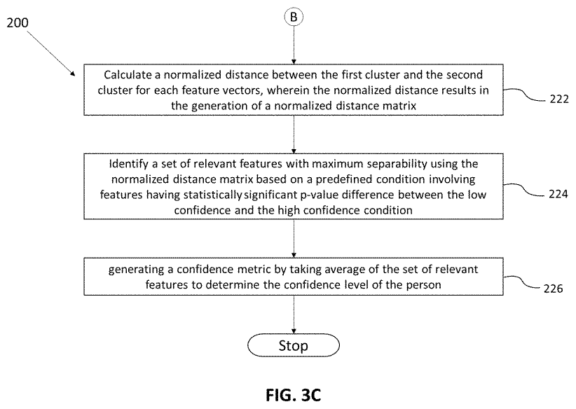

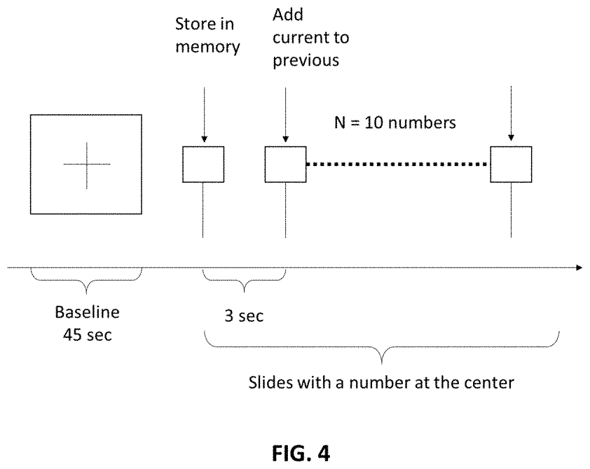

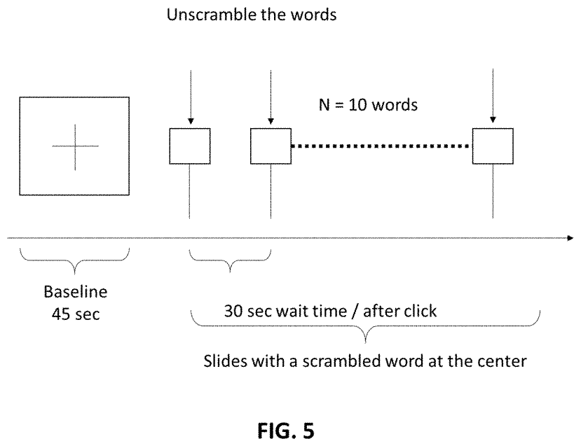

In operation, a flowchart 200 illustrating a method for determining confidence level of the person using the electroencephalogram (EEG) is shown in FIG. 3A-3C. Initially at step 202, a stimulus is provided to the person using the display screen 102 present in front of the person. In an example of the disclosure, two types of stimulus are provided, the addition task and the anagram task as their schema is shown in FIG. 4 and FIG. 5 respectively. Both the tasks are designed with high cognitive load and low cognitive load. At the next step 204, the electroencephalogram (EEG) signal of the person is captured in response to the stimulus using the EEG sensor 104 attached on the person, wherein the EEG sensor 104 is using a predefined number of electrodes for sensing. In an example, the EEG headband 134 from Muse is used as the EEG sensor 104. It has four dry electrodes corresponding to Fp1, Fp2, TP9 and TP10 with sampling frequency of 220 Hz.

Further, the signal processing is performed on the captured EEG signal to remove various noise and artifacts. At step 206, the captured EEG signal is filtered using the 4th order Butterworth band pass filter. Followed by at step 208, the independent component analysis (ICA) is performed on the filtered EEG signal to remove the artifacts generated due to eye blink of the person. The independent components can be visually inspected and the component from the eye blink can be removed. In the next step 210, the filtered EEG signal is reconstructed after removing the artifacts.

In the next step, the reconstructed EEG signal is decomposed into three frequency bands namely, theta (4.5-7.5 Hz), alpha (8-12.5 Hz) and beta (13-30 Hz). At step, 214, the band powers are calculated corresponding to each of the three frequency bands. The band powers are calculated taking a 500 ms long epoch before the end time-point of each trial. At step 216, the band power feature vector is generated corresponding to a plurality of elements. The plurality of elements are decided based on the predefined number of electrodes and the three frequency bands. In the present example, 4 electrodes have been used for three frequency bands, so the number of elements will be 12. The generated band power feature vectors are represented in a feature matrix;

In the next step 218, outliers are removed from the feature matrix using a random sample consensus (RANSAC) method, wherein the removal results in the generation of an inliers feature matrix. Further at step 220, a first cluster corresponding to a high confidence condition and a second cluster corresponding to a low confidence condition are determined for the each of the feature vectors of the inlier feature matrix. At step 222, the normalized distance between the first cluster and the second cluster for each feature vectors is calculated. The calculation of the normalized distance results in the generation of the normalized distance matrix (Xn).

In the next step 224, the set of relevant features with maximum separability using the normalized distance matrix are identified based on a predefined condition involving features having statistically significant p-value difference between the low confidence and the high confidence condition. The predefined condition is the normalized distance matrix values corresponding to low confidence is between 0 to 0.5 and the normalized distance matrix values corresponding to high confidence is between 0.5 to 1.0. And finally at step 226, the confidence metric is generated by taking average of the set of relevant features. The generated confidence metric is then used to determine the confidence level of the person.

According to an embodiment of the disclosure, the system 100 can also be explained with the help experimental procedures and results. In the present example, the low cost EEG headband from Muse has been used. It has four dry electrodes corresponding to Fp1, Fp2, TP9, TP10 with sampling frequency of 220 Hz. The reference electrode is at Fpz and DRLs are at a distance of one inch from the reference on both sides. The headband is worn like a pair of glasses, with the frontal electrodes placed over the middle of the forehead, and the rear electrodes placed behind the ears as shown in the experimental setup of FIG. 2. The stimulusis presented in a LCD computer screen (resolution 1366*768 pixels) placed at a distance of 60 cm from the participant.

Two stimulus (addition and anagram task) have been used to study the relationship between CL and the confidence level. The schema of addition task and the anagram task is shown in FIG. 4 and FIG. 5 respectively.

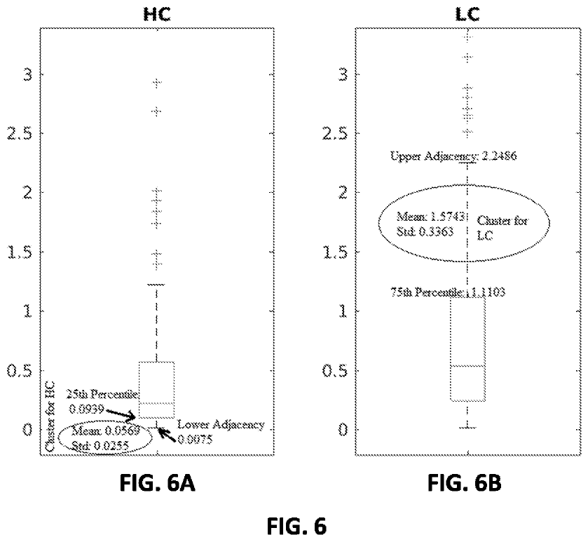

The following example explains how the clusters for HC and LC are determined from the boxplot. FIG. 6A and FIG. 6B is depicting the boxplot of feature Theta_Fp2 for HC and LC respectively. First the median of the HC and LC boxplots are compared. If median_HC>median_LC then the upper-whisker of HC is used for HC_cluster and the lower-whisker of LC is used for LC_cluster, otherwise if median_LC>median_HC then the lower-whisker of HC is used for HC_cluster and the upper-whisker of LC is used for LC_cluster. Median_LC>median_HC, therefore the lower-whisker of HC i.e. 0.0169 (25th Percentile) to 0.0003 (Lower Adjacency) is used for HC cluster and upper-whisker of LC i.e. 0.4512 (75th Percentile) to 1.0152 (Upper Adjacency) was used for LC cluster. The mean and standard deviation of the data, fallen between these lower/upper whisker ranges are taken as the cluster mean and standard deviation. Thus for the current example for feature Theta_Fp2, the cluster mean±std for HC is 0.0101±0.0045 and the cluster mean±std for HC is 0.6514±0.1863. Thus the cluster mean±std for both the classes (HC and LC) is calculated for all the 12 features (Theta_TP9, Theta Fp1, . . . , Beta Fp10, TP10). This is shown in Table 2 and its pictorial representation is shown in FIG. 7.

TABLE 1
The mean and standard deviation of each cluster
corresponding to each feature type

| Feature | | | | | |
|---|---|---|---|---|---|
| Seq | Features | Mean | Std | Mean | Std |
| 1 | Theta_TP9 | 1.0795 | 0.2615 | 0.0242 | 0.0135 |
| 2 | Theta_Fp1 | 0.3747 | 0.0914 | 0.021 | 0.0114 |
| 3 | Theta_Fp2 | 0.0101 | 0.0045 | 0.6514 | 0.1863 |
| 4 | Theta_TP10 | 0.7692 | 0.1713 | 0.0287 | 0.0122 |
| 5 | Alpha_TP9 | 0.2196 | 0.0867 | 2.0138 | 0.4561 |
| 6 | Alpha_Fp1 | 0.1 | 0.0479 | 2.0123 | 0.4788 |
| 7 | Alpha_Fp2 | 0.0569 | 0.0255 | 1.5743 | 0.3363 |
| 8 | Alpha_TP10 | 2.0323 | 0.5541 | 0.092 | 0.0536 |
| 9 | Beta_TP9 | 1.9915 | 0.3819 | 0.3236 | 0.2037 |
| 10 | Beta_Fp1 | 1.9124 | 0.3111 | 0.3441 | 0.1031 |
| 11 | Beta_Fp2 | 2.0272 | 0.3064 | 0.5047 | 0.1023 |
| 12 | Beta_TP10 | 0.4082 | 0.0754 | 1.7385 | 0.3475 |

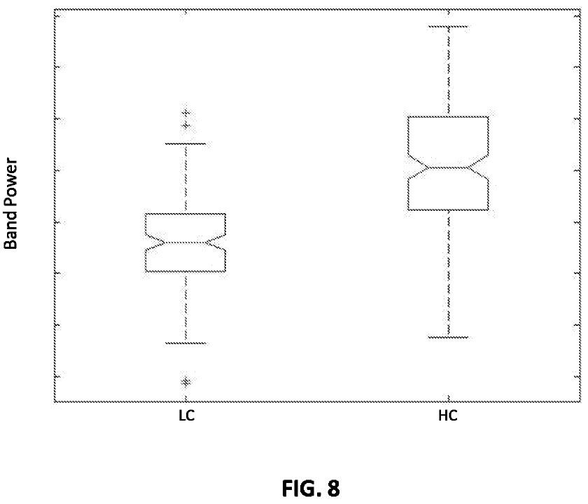

Further the normalized distance matrix Xn is determined (dim: Mx12) corresponding to HC and LC where it is expected in ideal case that all the Xn values related to HC should be >0.5 and Xn values related to LC should be <0.5. The two-sample t-test between HC and LC based on the CFM formulation given in equation (3) yields a p-value<0.05 and the classification accuracy against the threshold 0.5 is 73.79%. The separation between the distributions of the HC and LC are shown in FIG. 8.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein solves the difficulty of detection of confidence level of the person using electroencephalogram of the person. The disclosure provides a method and system for determining confidence level using the EEG generated captured from the person in response to a stimulus.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software modules located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope and spirit of disclosed embodiments being indicated by the following claims.

What is claimed is:

1. A method (200) for determining confidence level of a person using electroencephalogram (EEG), the method comprising a processor implemented steps of:
providing a stimulus to the person using a display screen present in front of the person (202);
capturing electroencephalogram (EEG) signal of the person in response to the stimulus using an EEG sensor attached on the person (204), wherein the EEG sensor is using a predefined number of electrodes for sensing;
filtering the captured EEG signal using a band pass filter (206);
performing an independent component analysis (ICA) on the filtered EEG signal to remove the artifacts generated due to eye blink of the person (208);
reconstructing the filtered EEG signal after removing the artifacts (210);
decomposing the reconstructed EEG signal into three frequency bands (212);
calculating band powers corresponding to each of the three frequency bands (214);
generating band power feature vectors corresponding to each of a plurality of elements, wherein the plurality of elements are decided based on the predefined number of electrodes and the three frequency bands, wherein the generated band power feature vectors are represented in a feature matrix (216);
removing outliers from the feature matrix using a random sample consensus (RANSAC) method, wherein the removal results in the generation of an inliers feature matrix (218);
determining a first cluster corresponding to a high confidence condition and a second cluster corresponding to a low confidence condition for the each of the feature vectors of the inlier feature matrix (220);
calculating a normalized distance between the first cluster and the second cluster for each feature vectors, wherein the normalized distance results in the generation of a normalized distance matrix (222);
identifying a set of relevant features with maximum separability using the normalized distance matrix based on a predefined condition involving features having statistically significant p-value difference between the low confidence and the high confidence condition (224); and
generating a confidence metric by taking average of the set of relevant features to determine the confidence level of the person (226).

2. The method of claim 1, wherein the predefined condition is the normalized distance matrix values corresponding to low confidence is between 0 to 0.5 and the normalized distance matrix values corresponding to high confidence is between 0.5 to 1.0.

3. The method of claim 1 further comprising the step of calculating F1 score and the p-value from a two-sample t-test to determine the quality of separation.

4. The method of claim 1 further comprising the step of calculating the accuracy of the predicted confidence level of the person.

5. The method of claim 1 wherein the EEG sensor is using four measuring electrodes and one neutral electrode for sensing the EEG signal of the person.

6. The method of claim 1, wherein the band pass filter is filtering the captured EEG signal with a pass band between 0.5 Hz to 40 Hz.

7. The method of claim 1, wherein the three frequency bands are theta, alpha and beta, wherein the range of theta is about 4.5 to 7.5 Hz, the range of alpha is about 8 to 12.5 Hz and the range of beta is about 13 to 30 Hz.

8. The method of claim 1, wherein the step of providing stimulus includes providing an addition task and an anagram task.

9. The method of claim 1 further comprising the step of calculation of an un-mixing matrix for obtaining the independent components.

10. A system (100) for determining confidence level of a person using electroencephalogram (EEG), the system comprises
a display screen (102) present in front of the person to provide a stimulus;
an EEG sensor (104) attached on the person configured to capture electroencephalogram (EEG) signal of the person in response to the stimulus, wherein the EEG sensor is using a predefined number of electrodes for sensing;
a memory (106); and
a processor (108) in communication with the memory, wherein the processor further comprises:
a filtering module (110) configured to filter the captured EEG signal using a band pass filter;
an independent component analysis module (112) for performing an independent component analysis (ICA) on the filtered EEG signal to remove the artifacts generated due to eye blink of the person;
a reconstruction module (114) for reconstructing the filtered EEG signal after removing the artifacts;
a decomposing module (116) for decomposing the reconstructed EEG signal into three frequency bands;
a band power calculation module (118) for calculating band powers corresponding to each of the three frequency bands;
a band power feature vector generation module (120) for generating band power feature vectors corresponding to each of a plurality of elements, wherein the plurality of elements are decided based on the predefined number of electrodes and the three frequency bands, wherein the generated band power feature vectors are represented in a feature matrix;
an outlier removal module (122) for removing outliers from the feature matrix using a random sample consensus (RANSAC) method, wherein the removal results in the generation of an inliers feature matrix;
a cluster determination module (124) for determining a first cluster corresponding to a high confidence condition and a second cluster corresponding to a low confidence condition for the each of the feature vectors of the inlier feature matrix;
a normalized distance calculation module (126) for calculating a normalized distance between the first cluster and the second cluster for each feature vectors, wherein the normalized distance results in the generation of a normalized distance matrix;
a relevant feature identification module (128) for identifying a set of relevant features with maximum separability using the normalized distance matrix based on a predefined condition involving features having statistically significant p-value difference between the low confidence and the high confidence condition; and
a confidence metric generation module (130) for generating a confidence metric by taking average of the set of relevant features to determine the confidence level of the person.

11. A computer program product comprising a non-transitory computer readable medium having a computer readable program embodied therein, wherein the computer readable program, when executed on a computing device, causes the computing device to:
provide a stimulus to the person using a display screen present in front of the person;
capture electroencephalogram (EEG) signal of the person in response to the stimulus using an EEG sensor attached on the person, wherein the EEG sensor is using a predefined number of electrodes for sensing;
filter the captured EEG signal using a band pass filter;
perform an independent component analysis (ICA) on the filtered EEG signal to remove the artifacts generated due to eye blink of the person(208);
reconstruct the filtered EEG signal after removing the artifacts;
decompose the reconstructed EEG signal into three frequency bands;
calculate band powers corresponding to each of the three frequency bands;
generate band power feature vectors corresponding to each of a plurality of elements, wherein the plurality of elements are decided based on the predefined number of electrodes and the three frequency bands, wherein the generated band power feature vectors are represented in a feature matrix;
remove outliers from the feature matrix using a random sample consensus (RANSAC) method, wherein the removal results in the generation of an inliers feature matrix;
determine a first cluster corresponding to a high confidence condition and a second cluster corresponding to a low confidence condition for the each of the feature vectors of the inlier feature matrix;
calculate a normalized distance between the first cluster and the second cluster for each feature vectors, wherein the normalized distance results in the generation of a normalized distance matrix;
identify a set of relevant features with maximum separability using the normalized distance matrix based on a predefined condition involving features having statistically significant p-value difference between the low confidence and the high confidence condition; and
generate a confidence metric by taking average of the set of relevant features to determine the confidence level of the person.
